(19) United States
(12) Patent Application Publication
(10) Pub. No.: US 2010/0166662 A1
(43) Pub. Date: Jul. 1, 2010

(54) MAGNETIC RESONANCE IMAGING METHOD USING VANADYL-BASED CONTRAST AGENTS

(75) Inventors: Devkumar Mustafi, Chicago, IL (US); Gregory S. Karczmar, Crete, IL (US); Marvin W. Makinen, Chicago, IL (US); Marta Zamora, Morris, IL (US); Edward S. Foxley, Chicago, IL (US)

Correspondence Address: MICHAEL BEST & FRIEDRICH LLP, 100 East Wisconsin Avenue, Suite 3300, Milwaukee, WI, 53202, US

(73) Assignee: The University of Chicago, Chicago, IL (US)

(21) Appl. No.: 12/294,649

(22) PCT Filed: Mar. 27, 2007
(86) PCT No.: PCT/US07/07690

§ 371 Date: Sep. 26, 2008

Related U.S. Application Data
(60) Provisional application No. 60/786,276, filed on Mar. 27, 2006.

(51) Int. Cl. A61K 49/06; C07F 9/00

(52) U.S. Cl. 424/9.3; 556/42

(57) ABSTRACT

A new, clinically applicable magnetic resonance imaging (MRI) method has been developed for in vivo imaging of a population of cells in a subject based on a class of paramagnetic divalent vanadyl-based contrast agents. The method includes administering to a subject a V^{O2+}-based contrast agent and monitoring distribution of the V^{O2+}-based contrast agent in the subject using magnetic resonance imaging.

### CROSS-REFERENCE TO RELATED APPLICATIONS

This application claims priority to U.S. Provisional Patent Application No. 60/786,276 filed Mar. 27, 2006, which is incorporated by reference in its entirety.

### STATEMENT REGARDING FEDERALLY SPONSORED RESEARCH OR DEVELOPMENT

This invention was made with government support under 1 R01 EB003108-02 awarded by the National Institute of Health (NIH). The government has certain rights in this invention.

### INTRODUCTION

Early detection of cancer enhances the chances of successful treatment. Tumors embedded in tissue or an organ present a particular challenge to early detection. Some, but not all, of these tumors may be detected using traditional imaging techniques. Thus, there is a need in the art for new methods of imaging tumors.

### SUMMARY OF THE INVENTION

The present invention provides a method of in vivo imaging a population of cells in a subject including administering a VO²⁺-based contrast agent to the subject and monitoring distribution of the VO²⁺-based contrast agent in the subject using magnetic resonance imaging.

The present invention also provides a method of detecting cancer cells in a subject including obtaining a first magnetic resonance image of a region of the subject, administering a VO²⁺-based contrast agent to the subject, obtaining a second magnetic resonance image of the region, and comparing the first and second magnetic resonance images.

The present invention further provides a method of detecting cancer cells in a subject including administering a VO²⁺-based contrast agent to the subject, obtaining a magnetic resonance image of the subject, and identifying regions of enhanced contrast in the magnetic resonance image.

The present invention also provides a method of enhancing magnetic resonance imageability of a subject including administering to the subject an amount of a VO²⁺-based contrast agent effective to enhance a magnetic resonance image.

In addition, the present invention provides a composition including a VO²⁺-based contrast agent and a physiologically acceptable organic solvent and a physiologically acceptable buffer. The present invention further provides a composition including a VO²⁺-based contrast agent, and a physiologically acceptable carrier, wherein the VO²⁺-based contrast agent is present in a concentration of at least about 1 mM.

The present invention provides a VO²⁺-based contrast agent having the formula

VO[(Lₘ)ₙ₁(L_{b})ₙ₂(Lₜ)ₙ₃(Lₑ)ₙ₄]

wherein
Lₘ is a monodentate organic ligand;
L_{b} is a bidentate organic ligand;
Lₜ is a tridentate organic ligand; and
Lₑ is a tetradentate organic ligand; and
   wherein
n₁=0, 1, 2, 3 or 4;
n₂=0, 1 or 2;
n₃=0 or 1; and
n₄=0 or 1;
   such that
when n₁=4, then n₂, n₃ and n₄=0;
when n₁=2 and n₂=1, then n₃ and n₄=0;
when n₁=1 and n₃=1, then n₂ and n₄=0;
when n₂=2 then n₁, n₃ and O₄=0; and
when n₄=1 then n₁, n₂ and n₃=0.

The present invention also provides VO²⁺-based contrast agent having one of the following formulas:

VO(Lₘ¹Lₘ²Lₘ³Lₘ⁴),

VO(Lₘ¹Lₘ²L_{b}¹),

VO(L_{b}¹L_{b}²),

VO(LₜLₘ¹), or

VO(Lₑ)

wherein Lₘ¹, Lₘ², Lₘ³ and Lₘ⁴ are the same or different monodentate ligands selected from the group consisting of a heteroalkyl group, a substituted heteroalkyl group, a substituted alkyl group, a substituted aromatic group, a substituted heteroaromatic group, a substituted carbocyclic group, a substituted heterocyclic group, wherein each ligand is bound to vanadium through a coordinating atom selected from the group consisting of oxygen and sulfur; and

wherein L_{b}¹ and L_{b}² are the same or different bidentate ligands selected from the group consisting of a heteroalkyl group, a substituted heteroalkyl group, a substituted alkyl group, a substituted aromatic group, a substituted heteroaromatic group, a substituted carbocyclic group, a substituted heterocyclic group, wherein each ligand is bound to vanadium through two coordinating atoms selected from the group consisting of oxygen, sulfur and combinations thereof; and

wherein Lₜ is a tridentate ligand selected from the group consisting of a heteroalkyl group, a substituted heteroalkyl group, a substituted alkyl group, a substituted aromatic group, a substituted heteroaromatic group, a substituted carbocyclic group, a substituted heterocyclic group, wherein each ligand is bound to vanadium through three coordinating atoms selected from the group consisting of oxygen, sulfur and combinations thereof; and

wherein Lₑ is a tetradentate ligand selected from the group consisting of a heteroalkyl group, a substituted heteroalkyl group, a substituted alkyl group, a substituted aromatic group, a substituted heteroaromatic group, a substituted carbocyclic group, a substituted heterocyclic group, wherein each ligand is bound to vanadium through four coordinating atoms selected from the group consisting of oxygen, sulfur and combinations thereof.

The present invention also provides a VO²⁺ compound having the formula:

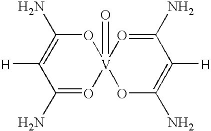

which may be of particular value in magnetic resonance imaging.

### BRIEF DESCRIPTION OF THE DRAWINGS

FIG. 1 is a schematic representation of insulin-mediated uptake of glucose, glycolysis, and gluconeogenesis in a cell.

FIG. 2 is a histogram plot of glucose uptake by 3T3-L1 adipocytes as a function of VO(acac)₂ concentration in the absence (open bars) and presence (closed bars) of 1 mM serum albumin. Glucose transport rates were measured with 2-deoxy-D-[1-¹⁴C]glucose. The vertical axis indicates glucose transport rates in units of pmol/min/well.

FIG. 3 shows two Western blots of p-AKT using Caco-2 and HCA-7 cells treated with 100, 250 and 500 μM VO(acac)₂ and compares them with controls.

FIG. 4 shows a VO(acac)₂-enhanced MR image of intracellular extracts (left panel) and spin-lattice relaxation time T₁-maps (right panel).

FIG. 5 shows atomic absorption measurements for samples of intracellular extracts with 250 and 500 μM VO(acac)₂, controls (intracellular extracts with no VO(acac)₂) and washings.

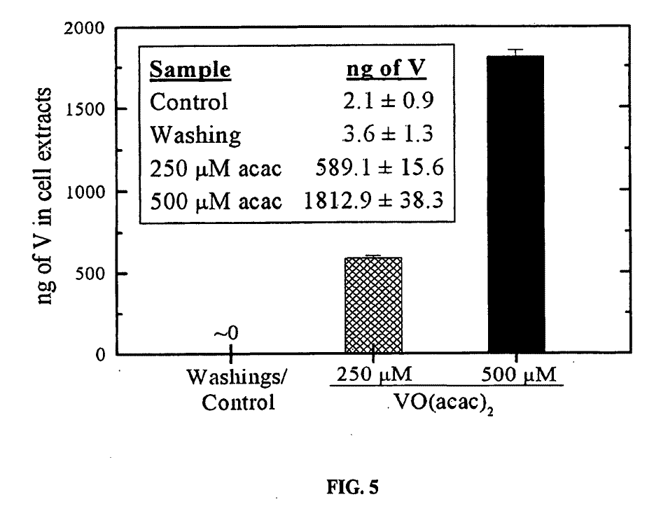

FIG. 6 shows high-resolution T₂*/T₁-weighted images of a rat bearing an AT6.1 tumor. Panel A shows a pre-contrast image; Panel B shows four reference (control) images, i.e., [pre-contrast]−[pre-contrast]; and Panel C shows difference MR images between the post-contrast and pre-contrast of the same tumor after administration of VO(acac)₂. The three rows in Panel C show the difference images up to about 2 hours.

FIG. 7 shows time series plots, i.e., the accumulation of the contrast agent in the tumor and surrounding muscles with time.

FIG. 8 shows the vanadyl concentration in tumor and muscle cells at various times after injecting mice with 0.15 mmol/kg of VO(acac)₂ contrast agent.

### DETAILED DESCRIPTION OF THE INVENTION

A new, clinically applicable magnetic resonance imaging (MRI) method has been developed for in vivo imaging of a population of cells in a subject based on a class of paramagnetic divalent vanadyl (VO²⁺)-based contrast agents. This new technique is suitable for imaging organs, tumors, cancer cells, etc. Suitably, the population of cells is selected from the group consisting of pancreas, brain, lung, kidney, thyroid, genitourinary, colorectal, gastrointestinal, liver, central nervous system, peripheral nervous system, prostate, heart and breast. The method of the present invention may be used to diagnosis cancers including, but not limited to lung cancer, prostate cancer, testicular cancer, brain cancer, skin cancer, colon cancer, rectal cancer, gastric cancer, esophageal cancer, tracheal cancer, head and neck cancer, pancreatic cancer, liver cancer, breast cancer, ovarian cancer, lymphoid cancer, leukemia, cervical cancer, vulvar cancer, melanoma, renal cancer, bladder cancer, thyroid cancer, bone cancers or soft tissue cancers.

Briefly, the imaging method involves administering a VO²⁺-based contrast agent to a subject and monitoring distribution of the VO²⁺-based contrast agent in the subject using magnetic resonance imaging. As used herein, “monitoring distribution of the VO²⁺-based contrast agent” refers to obtaining one or more magnetic resonance images of the subject. Suitably, a first magnetic resonance image may be obtained prior, to administering the VO²⁺-based contrast agent to the subject and a second magnetic resonance image may be obtained after administering the VO²⁺-based contrast agent to subject. The first and the second magnetic resonance images may then be compared. Suitably, the second magnetic image is obtained after a period of time has elapsed following the administration of the VO²⁺-based contrast agent. The time period may be at least about 0.25 minutes, at least about 0.5 minutes, at least about 1 minute, at least about 2 minutes, at least about 3 minutes, at least about 4 minutes, at least about 5 minutes, at least about 6 minutes, at least about 7 minutes, at least about 8 minutes, at least about 9 minutes, at least about 10 minutes, at least about 11 minutes, at least about 12 minutes, at least about 13 minutes, at least about 14 minutes, at least about 15 minutes, at least about 30 minutes, at least about 45 minutes, at least about 60 minutes, at least about 90 minutes, at least about 120 minutes, at least about 180 minutes, at least about 240 minutes, at least about 300 minutes, or at least about 360 minutes.

The subject is suitably a mammal, such as a mouse, rat, dog, cat, or human.

Suitably, the new method can be used to detect cancer cells. By selecting VO²⁺-based contrast agents that preferentially concentrate in cancer cells as compared to non-cancer cells, it is possible to enhance the magnetic resonance image of the cancer cells. Such contrast agents can facilitate the use of functional MRI to detect cancer at an early stage with increased diagnostic accuracy, to guide the design of optimal therapy for individual patients, and to lead to more efficient drug development.

The new method can also be used to detect cancer cells in a subject. A VO²⁺-based contrast agent is administered to the subject and a magnetic resonance image of the subject is obtained. Regions of enhanced contrast are then identified in the magnetic resonance image.

The new method can also be used to enhance magnetic resonance imageability of a subject. A VO²⁺-based contrast agent is administered to the subject in an amount effective to enhance a magnetic resonance image.

As used herein, and generally in the art, a “contrast agent” is one that enhances the contrast in an MRI image generated by introducing the agent in the zone being imaged. The enhanced contrast thus obtained enables particular organs or tissues to be visualized more clearly by increasing the MR signal of the particular organ or tissue relative to that of its surrounding environment. Most commonly, paramagnetic species are used to achieve a contrast effect.

Any VO²⁺ compound that (a) enhances T1 and/or T2* contrast in an MR image, and (b) preferentially concentrates in certain cells, for example, glycolytically active cells, such as cancer cells, may serve as a VO²⁺-based contrast agent. Without being bound by theory, it is thought that the preferential uptake of VO²⁺-based contrast agents by cancer cells relative to non-cancer cells results from the increased metabolic and/or glycolytic activity of cancer cells.

Of particular interest are contrast agents comprising VO²⁺ compounds having one or more organic ligands. The organic ligands may be monodentate, bidentate, tridentate or tetradentate or a combination thereof such that at least one coordination site remains open for a coordinated water molecule. The organic ligands are suitably coordinated to the vanadyl ion through at least one heteroatom, such as oxygen or sulfur. In addition, the organic ligands may contain one or more substituents having an exchangeable proton. Such substituents include, but are not limited to, amino, thiol and hydroxyl. Suitable organic ligands include, but are not limited to, acetylacetone, acetoacetamide, malonamide, 3-hydroxyl-2-methyl-4-pyrone and 2-mercaptopyridine-N-oxide. Suitably, the VO²⁺-based contrast agents of the present invention are substantially water-soluble.

Suitably, the VO²⁺-based contrast agent has the formula

VO[(Lₘ)ₙ₁(L_{b})ₙ₂(Lₜ)ₙ₃(Lₑ)ₙ₄]

wherein
Lₘ is a monodentate organic ligand;
L_{b} is a bidentate organic ligand;
Lₜ is a tridentate organic ligand; and
Lₑ is a tetradentate organic ligand; and
   wherein
n₁=0, 1, 2, 3 or 4;
n₂=0, 1 or 2;
n₃=0 or 1; and
n₄=0 or 1;
   such that
when n₁=4, then n₂, n₃ and n₄=0;
when n₁=2 and n₂=1, then n₃ and n₄=0;
when n₁=1 and n₃=1, then n₂ and n₄=0;
when n₂=2 then n₁, n₃ and n₄=0; and
when n₄=1 then n₁, n₂ and n₃=0.

More particularly, the VO²⁺-based contrast agent has the formula

VO(Lₘ¹Lₘ²Lₘ³Lₘ⁴),

VO(Lₘ¹Lₘ²L_{b}¹),

VO(L_{b}¹L_{b}²),

VO(LₜLₘ¹), or

VO(Lₑ)²⁺

wherein Lₘ¹, Lₘ², Lₘ³ and Lₘ⁴ are the same or different monodentate ligands selected from the group consisting of a heteroalkyl group, a substituted heteroalkyl group, a substituted alkyl group, a substituted aromatic group, a substituted heteroaromatic group, a substituted carbocyclic group, a substituted heterocyclic group, wherein each ligand is bound to vanadium through a coordinating atom selected from the group consisting of oxygen and sulfur; and

wherein L_{b}¹ and L_{b}² are the same or different bidentate ligands selected from the group consisting of a heteroalkyl group, a substituted heteroalkyl group, a substituted alkyl group, a substituted aromatic group, a substituted heteroaromatic group, a substituted carbocyclic group, a substituted heterocyclic group, wherein each ligand is bound to vanadium through two coordinating atoms selected from the group consisting of oxygen, sulfur and combinations thereof; and

wherein Lₜ is a tridentate ligand selected from the group consisting of a heteroalkyl group, a substituted heteroalkyl group, a substituted alkyl group, a substituted aromatic group, a substituted heteroaromatic group, a substituted carbocyclic group, a substituted heterocyclic group, wherein each ligand is bound to vanadium through three coordinating atoms selected from the group consisting of oxygen, sulfur and combinations thereof; and

wherein Lₑ is a tetradentate ligand selected from the group consisting of a heteroalkyl group, a substituted heteroalkyl group, a substituted alkyl group, a substituted aromatic group, a substituted heteroaromatic group, a substituted carbocyclic group, a substituted heterocyclic group, wherein each ligand is bound to vanadium through four coordinating atoms selected from the group consisting of oxygen, sulfur and combinations thereof.

As used herein, the term “alkyl group” means a chain of 1 to 18 carbon atoms. “Lower alkyl group” means an alkyl group having 1 to 6 carbon atoms. Exemplary alkyl groups include methyl, ethyl, propyl, and butyl. Alkyl groups may have a straight chain or branched chain structure. Alkyl groups may be saturated or unsaturated. Unsaturated alkyl groups have one or more double bonds, one or more triple bonds, or combinations thereof.

The term “aromatic group” means a monovalent group having a monocyclic ring structure or fused bicyclic or tricyclic ring structure. Monocyclic aromatic groups contain 5 to 10 carbon atoms, particularly 5 to 7 carbon atoms, and more particularly 5 to 6 carbon atoms in the ring. Bicyclic aromatic groups contain 7 to 17 carbon atoms, particularly 7 to 14 carbon atoms, and more particularly 9 or 10 carbon atoms in the ring. Exemplary aromatic groups include phenyl, naphthaline, and phenanthrene.

The term “carbocyclic group” means a monovalent saturated or unsaturated hydrocarbon ring. Carbocyclic groups are monocyclic, or are fused, Spiro, or bridged bicyclic ring systems. Monocyclic carbocyclic groups contain 4 to 10 carbon atoms, particularly 4 to 7 carbon atoms, and more particularly 5 to 6 carbon atoms in the ring. Bicyclic carbocyclic groups contain 7 to 17 carbon atoms, particularly 7 to 14 carbon atoms, and more particularly 9 to 10 carbon atoms in the ring. Monocyclic carbocyclic groups are not aromatic. However, bicyclic carbocyclic groups may contain one aromatic ring.

The term “heteroalkyl group” means a saturated or unsaturated chain containing 1 to 18 member atoms (i.e., including both carbon and at least one heteroatom). The chain may be straight or branched. Unsaturated heteroalkyl groups have one or more double bonds, one or more triple bonds, or both. Heteroalkyl groups are unsubstituted. Heteroalkyl groups may include ethers and thiols.

The term “heteroaromatic group” means an aromatic ring containing carbon and 1 to 4 heteroatoms in the ring. Heteroaromatic groups are monocyclic or fused bicyclic rings. Monocyclic heteroaromatic groups contain 5 to 10 member atoms (i.e., carbon and heteroatoms), particularly 5 to 7, and more particularly 5 to 6 member atoms. Bicyclic heteroaromatic rings contain 7 to 17 member atoms, particularly 7 to 14, and more particularly 9 or 10 member atoms. Heteroaromatic groups are unsubstituted. Heteroaromatic groups may include thienyl, thiazolyl, purinyl, pyrimidyl, pyridyl, and furanyl.

The term “heteroatom” means an atom other than carbon in the ring of a heterocyclic group or heteroaromatic group or the chain of a heteroalkyl group. Heteroatoms may be selected from the group consisting of nitrogen, sulfur, and oxygen atoms. Groups containing more than one heteroatom may contain different heteroatoms.

The term “heterocyclic group” means a saturated or unsaturated ring structure containing carbon and 1 to 4 heteroatoms in the ring. Heterocyclic groups are monocyclic, or are fused or bridged bicyclic ring systems. Monocyclic heterocyclic groups contain 4 to 10 member atoms (i.e., including both carbon atoms and at least 1 heteroatom), particularly 4 to 7, and more particularly 5 to 6 member atoms. Bicyclic heterocyclic groups contain 7 to 17 member atoms, particularly 7 to 14, and more particularly 9 or 10 member atoms. Monocyclic heterocylic groups are not aromatic. However, bicyclic heterocyclic groups may contain one aromatic ring. Heterocyclic groups are unsubstituted. Heterocyclic groups may include piperazinyl, morpholinyl, tetrahydrofuranyl, and piperidyl.

The term “substituted alkyl group” means an alkyl group wherein at least one of the hydrogen atoms bonded to a carbon atom in the chain has been replaced with another substituent.

The term “substituted aromatic group” means an aromatic group wherein at least one of the hydrogen atoms bonded to a carbon atom in the ring has been replaced with another substituent. The substituents may be substituted at the ortho, meta, or para position on the ring, or any combination thereof.

The term “substituted carbocyclic group” means a carbocyclic group wherein at least one of the hydrogen atoms bonded to a carbon atom in the ring has been replaced with another substituent.

The term “substituted heteroalkyl group” means a heteroalkyl group, wherein at least one of the hydrogen atoms bonded to a carbon atom in the chain has been replaced with another substituent. Exemplary substituted heteroalkyl groups include acetylamido and acetylacetanato.

The term “substituted heteroaromatic group” means a heteroaromatic group wherein 1 to 4 hydrogen atoms bonded to carbon atoms in the ring have been replaced with other substituents. Exemplary substituted heteroaromatic groups include 2-mercaptopyridine-N-oxide.

The term “substituted heterocyclic group” means a heterocyclic group wherein at least one of the hydrogen atoms bonded to a carbon atom in the ring has been replaced with another substituent. Exemplary substituted heteroaromatic groups include 3-hydroxyl-2-methyl-4-pyrone.

Substituents for the above groups may include halogen atoms, amino groups (e.g. amino, monoalkylamino, dialkylamino, alkylarylamino, diarylamino), hydroxyl groups, alkoxy groups (e.g. methoxy, ethoxy, propoxy, and butoxy), aryloxy groups, acyloxy groups; carbamoyloxy groups, carboxy groups, thiol groups (e.g., alkylthio groups, acythio groups, arylthio groups), cyano groups, oxo, alkyl groups, substituted alkyl groups, heteroalkyl groups, substituted heteroalkyl groups, aromatic groups, substituted aromatic groups, heteroaromatic groups, substituted heteroaromatic groups, phenoxy groups, or any combination thereof.

A novel VO²⁺ compound having the formula:

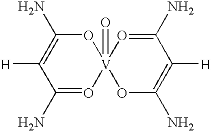

is also provided by the present invention. This compound may be of particular value as a MRI contrast agent.

Suitably, the VO²⁺-based contrast agent has a molecular weight of less than about 600, or less than about 500, or less than about 400, or less than about 300.

The VO²⁺-based contrast agent may be administered by any suitable method. Suitable methods include, but are not limited to, parenteral administration, such as intravenous, enteral administration, such as oral, and administration via inhalation. For example, the VO²⁺-based contrast agent may be administered into the vascular system. Alternatively, the VO²⁺-based contrast agent may be administered into the population of cells being imaged or directly into the vessels of a specific organ such as the coronary artery. The VO²⁺-based contrast agent is suitably administered over a time period of about 0.5 minutes to about 2 hours.

Suitably, the VO²⁺-based contrast agent is administered in an amount that is greater than about 0.01 mmol/kg body weight, more particularly greater than about 0.07 mmol/kg body weight, and even more particularly greater than about 0.10 mmol/kg body weight. The VO²⁺-based contrast agent is suitably administered in an amount that is less than about 0.20 mmol/kg body weight, more particularly less than about 0.15 mmol/kg body weight, and even more particularly less than about 0.10 mmol/kg body weight. This includes embodiments where the VO²⁺-based contrast agent dosage is about 0.1-0.2 mmol/kg body weight, and further includes embodiments where the VO²⁺-based contrast agent dosage is about 0.1-0.15 mmol/kg body weight.

Suitably, the VO²⁺-based contrast agent is administered in a pharmaceutical formulation. The pharmaceutical formulation may contain a physiologically acceptable carrier. The physiologically acceptable carrier may be a physiologically acceptable buffer or an isotonic solution. Suitable carriers include HEPES/NaCl and phosphate buffered saline (PBS)/NaCl. The formulation may also include a physiologically acceptable organic solvent, such as ethanol. The amount of physiologically acceptable organic solvent may be present in about 1% to about 10% by weight of the resulting formulation. Suitably, the concentration of the VO²⁺-based contrast agent formulation ranges from about 1 mM to about 10 mM. More particularly, the formulation is at least about 3 mM. Even more particularly, the formulation is at least about 5 mM. More particularly, the formulation is at least about 10 mM.

Without being bound by theory, it is thought that vanadyl-based contrast agents have a high specificity for glycolytically active cells. However, it is their specificity for glycolytically active cells coupled with their imaging properties that makes them ideal MR imaging probes. Both of these properties are discussed in more detail below.

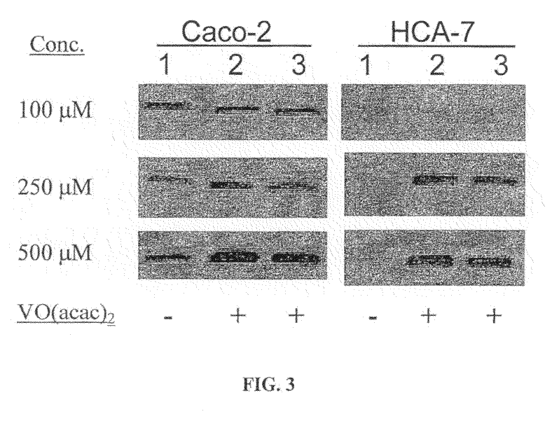

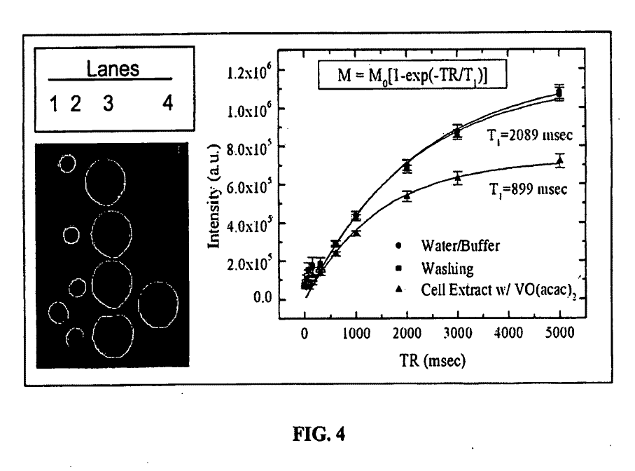

Vanadyl-based contrast agents that preferentially concentrate in certain cells have a number of advantages. Vanadyl-based contrast agents have very low toxicity, and thus can be used repeatedly at concentrations that produce strong MRI contrast. Many MRI contrast enhancement agents currently available suffer from toxicity, especially renal toxicity. The agents in accordance with the present invention accumulate in cells and are distributed intracellularly, resulting in a relatively strong, durable enhancement. Vanadyl-based contrast agents in accordance with the present invention interact strongly with intracellular receptor proteins (e.g., insulin receptor substrate-1), glycolytic enzymes, and possibly with glucose transporters. The abundance of these proteins provides a large number of intracellular binding sites for vanadyl. Vanadyl-based contrast agents in accordance with the present invention are sensitive to cancer metabolism so that they can help to identify cancers that have a high rate of metabolism and are growing rapidly. This is unusual for MRI contrast agents, since there are only a few reports of MRI contrast agents that are sensitive to metabolic rate. Vanadyl-based contrast agents in accordance with the present invention strongly bind to albumin in the blood, increasing the blood half-life of the contrast agent and improving its access to glycolytically active cells. In addition, vanadyl-based contrast agents in accordance with the present invention provided excellent T₁ and T₂* contrast, as shown in FIGS. 3 and 4. These properties are discussed in greater detail below.

The vanadyl (VO²⁺) ion or oxovanadium(IV) ion is one of the most stable diatomic ions known. In the ground state of vanadium(IV), the unpaired electron occupies the 3d_{xy} orbital, making it a sensitive paramagnetic probe for magnetic resonance spectroscopic studies and an efficient contrast agent. Vanadium is a trace element found naturally in biomineralization processes, including tooth and bone formation. Recent studies have suggested that organic chelates of vanadyl ions exhibit relatively low toxicity. For example, introduction of an organic chelate of the vanadyl ion into Phase I trials by Kinetek Pharmaceuticals, Inc., Vancouver, British Columbia, Canada, for treatment of diabetes has demonstrated that these compounds can be used clinically in relatively large concentrations with low toxicity. Recently, D. T. Puerta, et al. noted that chelators, such as maltol, have been found to form metal complexes with good aqueous solubility and biocompatibility. Puerta, D. T. et al., “Tris(pyrone) Chelates of Gd(III) as High Solubility MRI-CA,” J. Am. Chem. Soc., 128 (2006), p. 2222. For example, [V=O(maltolato)₂] has been widely studied as a soluble therapeutic vanadate source for treating type II diabetes. Thompson, K. H.; Orvig, C., Metal Ions Biol. Syst. 2004, 41, 221-252.

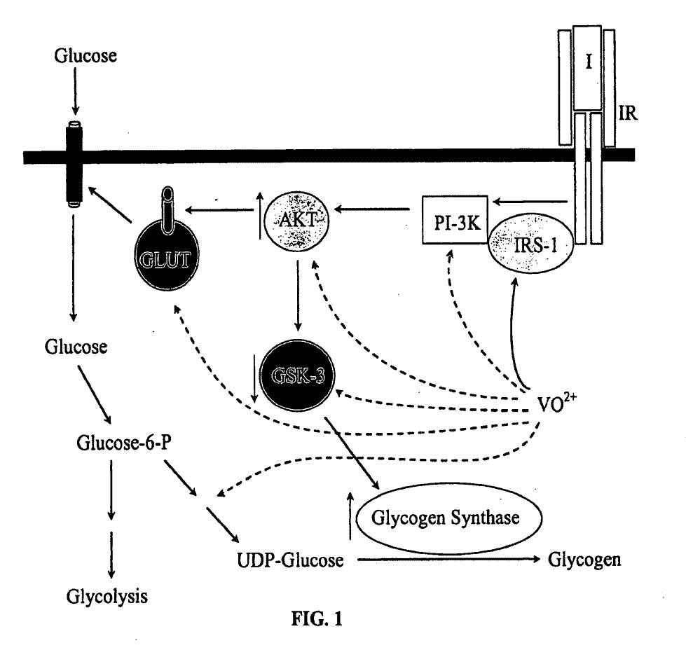

It is believed the vanadyl ion stimulates glycogen synthesis and PI-3K without any change in the tyrosine phosphorylation of the insulin receptor. It has previously been shown that in contrast to insulin, the vanadyl ion is associated with increased tyrosine phosphorylation of proteins, such as the β-subunit of the insulin receptor (IRβ) and the insulin receptor substrate-1 (IRS-1) inside the cell. H. Ou, L. Yan, D. Mustafi, M. W. Makinen, and M. J. Brady, “The vanadyl (VO²⁺) chelate bis(acetylacenato)oxovanandium(IV) potentiates tyrosine phosphorylation of the insulin receptor,” J. Biol. Inorg. Chem. 10, 874-886 (2005), which is incorporated by reference herein. The vanadyl ion stimulates glycogen synthesis and glucose uptake inside the cell in a manner that is independent of the very first step of the insulin pathway outside the cell, as shown in FIG. 1.

It has also been suggested that vanadyl compounds induce activation of AKT and enhance uptake of glucose into cells through glucose transporters. Vanadyl compounds induce changes in intracellular, phospho-active AKT (protein kinase B) and ERK (extracellular signal receptor kinase) in whole cell lysates. Both AKT and ERK are intracellular kinases. AKT plays a key role in many cellular processes such as glucose metabolism, cell proliferation, apoptosis, transcription and cell migration. The results of cell signaling experiments suggest that VO²⁺ compounds induce changes in key intracellular, glycolytically-active proteins and this may be due to intracellular uptake of the VO²⁺ compounds.

Based on studies of insulin-like actions of vanadyl compounds and their interactions with adipocytes in diabetic rats, it has been demonstrated that VO²⁺ is taken up by cells where it promotes insulin-like effects by activating glucose transporters. These studies strongly suggest that vanadyl compounds bind to (receptor) enzymes associated with glycolysis and glucose transport inside the cell. Because of the intimate association of VO²⁺ with the glycolytic apparatus, as shown in FIG. 1, the VO²⁺-based contrast agents in accordance with the present invention concentrate preferentially inside glycolytically active cells. This concentration of a contrast agent within the cells specifically enhances these cells in MR imaging and provides a new direction for development of contrast agents that can be used for detecting image specific molecular events in vivo.

In addition, these VO²⁺ compounds exhibit significantly enhanced insulin-mimetic activity in diabetic laboratory animals or adipocyte cells over that of inorganic VO²⁺ introduced as VOSO₄. Furthermore, because the capacity of organic ligand chelates of VO²⁺ to lower blood glucose of diabetic laboratory animals is equivalent whether administered gastrointestinally or intraperitoneally, the enhanced insulin-mimetic action compared with that of VOSO₄ could not be ascribed simply to increased lipophilic character of the organic ligand facilitating transport across the intestinal wall. These observations suggest that the structure of the organic chelating ligand and its electronic, i.e., bonding interactions with the VO²⁺ moiety are important factors directing the reactivity of VO²⁺ compounds with macromolecules involved in glycolysis. These properties can be harnessed through ligand design to improve specificity of action. Stimulation of glucose uptake by vanadyl is consistent with vanadyl having strong interactions with glycolytic enzymes. This is the basis for selective enhancement of glycolytically active cells in MR images.

As shown in the examples below, VO²⁺ compounds bind to albumin in the blood stream. Binding to serum albumin, for instance, as the major serum transport protein, may stabilize VO²⁺ against oxidation or result in formation of a specific [protein:VO²⁺ compound] adduct that is recognized at the membrane surface of target cells. Interaction with albumin should also increase the blood half-life of the VO²⁺ compounds, and thus increase the probability that they will reach the targeted cell population. In addition, due to binding to the macromolecules, the vanadyl will tend to leak preferentially into tumors through hyperpermeable tumor vasculature, as suggested by MRI results. This will facilitate access of the vanadyl ion to glycolytic tumor cells.

The T₁ relaxation times of VO(acac)₂ in 0.01 M HEPES buffer at pH 7.4 with 0.15 M NaCl were determined. With the determined T₁ values at different concentrations of VO(acac)₂, the relaxivity was determined using equation 1,

r1=[1/C](1/T₁ₘ−1/T_{1w}) (1)

where T₁ₘ is the measured T₁ for VO(acac)₂, T_{1w} is the T₁ for water, C is the concentration of the contrast agent, and r1 is the relaxivity. The relaxivity for VO(acac)₂ of 2.5±0.2 mM⁻¹s⁻¹ is slightly lower but close to that of known contrast agents of gadolinium complexes of 4.3±0.2 mM⁻¹s⁻¹ at 4.7 Tesla. This means that VO(acac)₂ can produce significant, easily detectable changes in MR image intensity.

It is to be understood that the invention is not limited in its application to the details of construction and the arrangement of components set forth in the description or illustrated in the drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. It also is understood that any numerical range recited herein includes all values from the lower value to the upper value. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this application.

Thus, the invention provides, among other things, a clinically applicable MRI method for imaging a population of cells based on a class of paramagnetic divalent vanadyl-based contrast agents that target receptor proteins in glycolysis. Various features and advantages of the invention are set forth in the claims.

The following Examples are provided to assist in a further understanding of the invention. The particular materials, methods and conditions employed are intended to be illustrative of the invention and are not limiting upon the scope of the invention.

### EXAMPLES

### Example 1

### Synthesis of Oxovanadium(IV) MRI Contrast Agents

Bis(acetylamido)oxovanadium(IV) [VO(acac-NH₂)₂]. VO(acac-NH₂)₂ was synthesized according to the procedure described by Crans et al., “Bis(acetylamido)oxovanadium(IV) Complexes: Solid State and Solution Studies,” J. Chem. Soc. Dalton Trans. (2001) pp. 3337-3345. One equivalent of vanadyl sulfate was dissolved in water followed by the addition of 3-equivalents of acetoacetamide under constant stirring. A solution of 10% sodium bicarbonate was then added dropwise to raise the pH from 1.5 to 3.6 in order to form a suspension. A bluish precipitate was obtained after allowing the solution to stand for one day at 4° C. The precipitate was filtered off, washed with a small amount of chloroform, and dried under vacuum. VO(acac-NH₂)₂ was completely soluble in aqueous solutions at physiological pH. % Calculated (% found) analytical data: C, 35.96 (35.77); H, 4.53 (4.56); and N, 10.49 (10.41).

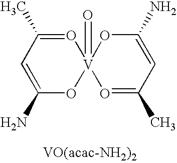

Bis(maltolato)oxovanadium(IV) [VO(malto)₂]. VO(malto)₂ was synthesized according to the procedure described by Caravan et al., “Reaction Chemistry of BMOV, Bis(maltolato)oxovanadium(IV)—A Potent Insulin Mimetic Agent,” J. Am. Chem. Soc. 117 (1995) pp. 12759-12770. One equivalent of vanadyl sulfate was dissolved in hot water followed by the addition of 2.1 equivalents of maltol (also known as 3-hydroxy-2-methyl-4-pyrone) under constant stirring. In order to bring the pH of the solution to ˜8.5, potassium hydroxide was added slowly over 2 hours. The resulting mixture was refluxed overnight, and upon cooling to room temperature, a birefringent purple/green solid was formed. The solid was collected by vacuum filtration, washed with cold water, and dried overnight in vacuo to obtain the desired product. The yield of VO(malto)₂ was about 58%.

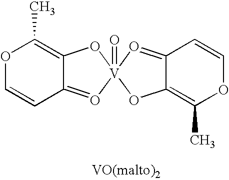

Bis(N-oxide-pyridine-2-thiolato)oxovanadium(IV) [VO(OPT)₂]. VO(OPT)₂ was synthesized according to the procedure described by Sakurai et al., “An Orally Active Vanadyl Complex, Bis(1-oxy-2-pyridinethiolato)oxovanadium(IV), with VO(S₂O₂) Coordination Mode, In Vitro and In Vivo Evaluations in Rats,” J. Inorg. Biochem. 80 (2000) pp. 99-105. 2-Mercaptopyridine-N-oxide was mixed with VOSO₄ in a 2:1 molar ratio of ligand:metal ion in an aqueous solution of pH˜6. The mixture was stirred at room temperature under nitrogen atmosphere for about 2 hours. Then the precipitate was collected, washed several times with water, and dried. The yield of VO(OPT)₂ was about 70%. %

Calculated (% found) analytical data: C, 37.61 (37.39); H, 2.51 (2.48); N, 8.78 (8.61). Mass spec revealed a m/e of 319.

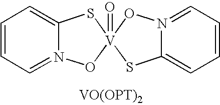

Bis(acetylacetanato-bisNH₂)oxovanadium(IV) [VO(acac-(NH₂)₂)₂]. VO(acac-(NH₂)₂)₂ is synthesized as follows. One equivalent of vanadyl sulfate is dissolved in water followed by the addition of 3 equivalents of malonamide under constant stirring. A solution of 10% sodium bicarbonate is then added dropwise to raise the pH in order to form a suspension. A bluish precipitate is obtained after standing the solution for one day at 4° C. The precipitate is filtered off, washed with a small amount of chloroform, and dried under vacuum. VO(acac-(NH₂)₂)₂ is completely soluble in aqueous solutions at physiological pH.

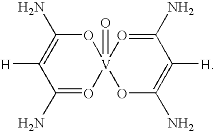

All chemicals were purchased from Sigma-Aldrich (Milwaukee, Wis.). De-ionized distilled water was use throughout. The desired products of VO(acac-NH₂)₂, VO(malto)₂ and VO(OPT)₂ were characterized by melting points, elemental analyses and mass spectrometry data.

### Example 2

### Vanadyl-Mediated Stimulation of Glucose Uptake and Metabolism

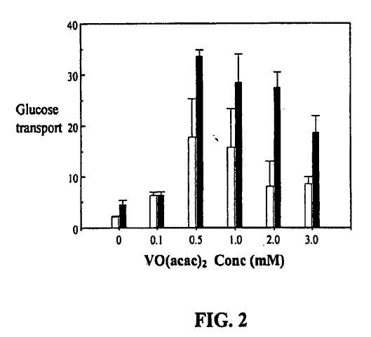

The effect of vanadyl compounds on uptake of 2-deoxy-D-[1-¹⁴C]glucose by 3T3-L1 adipocytes was measured using methods described in Makinen, M. W., et al., “Structural Origins of the Insulin-Mimetic Activity of Bis(acetylacetonato)oxovanadium(IV),” J. Biol. Chem. 277 (2002) pp. 12215-12220, which is incorporated herein by reference. Serum-starved, differentiated 373-L1 adipocytes were used in the metabolic assays. Cells were washed twice with phosphate-buffered isotonic saline at 37° C. and serum-starved for 2.5 hours in Krebs-Ringer buffer lacking bovine serum albumin or serum. The medium was removed, and cells were incubated for 30 minutes in buffer with different concentrations of VO²⁺-based contrast agents with or without serum albumin (1 mM) prior to measuring glucose uptake rates. Glucose transport rates were measured by the addition of 20 μM 2-deoxy-D-[1-¹⁴C] glucose. FIG. 2 is a histogram plot of glucose uptake by 3T3-L1 adipocytes as a function of VO(acac)₂ concentration in the absence (open bars) and presence (closed bars) of 1 mM serum albumin. As shown in FIG. 2, uptake of glucose was maximally enhanced by VO(acac)₂ in the presence of equimolar concentrations of serum albumin. Although VO(malto)₂ was observed to facilitate glucose uptake, there was no clear dependence of increase in glucose uptake on the VO(malto)₂:serum albumin ratio, and the enhancement per equivalent of VO²⁺ was markedly less than that observed for VO(acac)₂ and VO(OPT)₂ (data not shown).

### Example 3

### Cell Signaling Studies

HCA-7 and Caco-2 cells derived from human colonic adenocarcinomas obtained from the American Type Culture Collection, Manassas, Va. were cultured at 37° C. in a humidified atmosphere of 5% CO₂-95% air in 6-well plates (250,000 cells/well/2 mL in 5% serum medium). When cells were ˜70-80% confluent, cells were transferred to serum-free media and kept overnight. A vanadyl compound was added to a well to a final concentration of 100, 250, or 500 μM and incubated for 5 minutes. In each 6-well plate, 2 wells were set as controls and 4 wells were set for one desired concentration of a vanadyl compounds. Cells were then homogenized in 2× Laemmli sodium dodecyl sulfate (SDS) buffer. Samples were boiled for 5 minutes at 100° C. and proteins quantified using the RC-DC protein assay (Bio-Rad). Western blotting was then carried out, briefly described as follows. SDS-treated samples (50 μg/lane) were separated by SDS-polyacrylamide gel electrophoresis (PAGE) on a 4-15% resolving gel. Pre-stained molecular weight markers were included in each gel. Gels were electroblotted to immobilon-P transfer membranes (Millipore Billerica, Mass.). Following blocking of non-specific antibody binding, the blots were exposed to rabbit polyclonal anti-pAKT (Cell Signaling Technology, Danvers, Mass.) or mouse monoclonal anti-pERK (Santa Cruz Biotechnology, Inc., Santa Cruz, Calif.) and developed with secondary antibody using standard methods. The blots were washed, and the p-AKT and p-ERK were detected in film and digitized for final quantification.

FIG. 3 illustrates two Western blots of p-AKT using Caco-2 (left panel) and HCA-7 cells (right panel) treated with VO(acac)₂ in a concentration of 100, 250 or 500 μM (top, middle and bottom rows, respectively). Lane 1 is the control; lanes 2 and 3 are duplicates of cells exposed to VO(acac)₂ a particular concentration of VO(acac)₂. These blots were analyzed by xerography on X-OMAT™ autoradiogram films using an enhanced chemiluminesence system.

The results (duplicate measurements of n=3) showed that the level of p-AKT increased with increasing concentration of VO(acac)₂ (2-fold with 100 μM, 5-fold with 250 μM and 12-fold with 500 μM of VO(acac)₂ compared to controls). Similar results have been obtained with VO(acac)₂-induced activation of p-ERK. Similar results were also obtained with VO(maltolato)₂ and VO(OPT)₂. VOSO₄ did not induce measurable activation of p-AKT or p-ERK, possibly because at physiological pH, the inorganic VO²⁺ ion forms polymeric species. The results of cell signaling experiments, as described above, suggest that vanadyl chelates induce changes in key intracellular, glycolytically-active proteins and this may be due to intracellular uptake of vanadyl compounds.

### Example 4

### In Vitro Atomic Absorption and MRI Studies

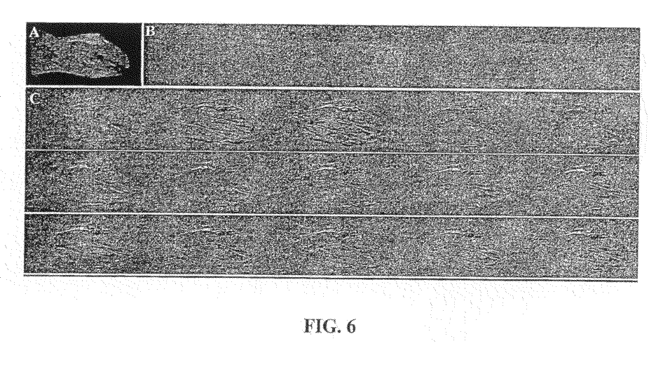

Evidence of the uptake of vanadyl compounds into cancer cells was found in MRI and atomic absorption studies carried out with intracellular extracts of HCA-7 cells after treating the cells with VO(acac)₂. The following protocol was used: HCA-7 cells were treated with 250 or 500 μM of VO(acac)₂, incubated for 10 minutes (based on the Western blot analysis of 2.5, 5, 10 and 30 minute time points), rinsed four times with phosphate buffered saline (PBS at pH=7.4), and then harvested with 1% trypsin. These concentrations were similar to what might be expected in vivo after a VO²⁺-based contrast agent is injected intravenously. The samples were re-suspended in 250 μL de-ionized (DI) water, sonicated for 60 sec, and centrifuged at 100,000×g. Then supernatants, representing only the cytosolic fraction, were collected for MR imaging and atomic absorption studies. The left panel in FIG. 4 shows a VO(acac)₂-enhanced MR image of intracellular extracts after treatment with 250 μM VO(acac)₂ in 500 μL tubes (Lane two, top two) and compares them to de-ionized water in a 1 mL tube (Lane 1), cell extracts from control experiments (Lane 2, bottom two), four washings in PBS buffer for samples as in Lane 2 (Lane 3) and PBS buffer (Lane 4). Samples for Lanes 3 and 4 were in 2 mL tubes. MR images were acquired using a Bruker scanner at 4.7 Tesla with the following parameters: echo time of 10.5 msec, field of view of 60 mm, array of 128×128, slice thickness of 3 mm, and with varying repetition times from 22 to 5000 msec. The right panel in FIG. 4 shows the spin-lattice relaxation time T₁-maps for water and buffer (black circles), washings (black squares) and cell extracts with VO(acac)₂ treatment (black triangles). The T₁-map of the cell extracts for the control experiments is identical to the washings and the buffer. The value of T₁ relaxation time of 899±149 msec for VO(acac)₂-treated cell extracts is significantly different (2089±234 msec) than that of cell extracts of controls, washings, DI water, or buffer. The decrease in T₁ time for samples of VO(acac)₂-treated cell extracts is indicative of significant concentration of vanadyl in cell extracts. FIG. 6 illustrates the amount of intracellular vanadium in HCA-7 cells measured by atomic absorption. The atomic absorption measurements were carried out for samples of intracellular extracts with 250 and 500 μM VO(acac)₂ and compared to that of controls (intracellular extracts with no VO(acac)₂) and washings using a Perkin Elmer AAnalyst 300 with an HGA furnace. The amount of V for each sample is shown in the inset (n=3). These measurements were based on standard curves of known vanadyl concentration with a correlation coefficient of 0.99828. The results demonstrate that there was a significant concentration of vanadium in the cell extracts, but none or very little in the washings. Thus, vanadyl was taken up in the cancer cells. Since atomic absorption is the most sensitive method of detecting metal ions, these results suggest that vanadyl compounds accumulated intracellularly.

### Example 5

### Toxicity of VO²⁺-Based Contrast Agents

Toxicity studies were performed with tumor-bearing nude mice weighing about 25-30 grams (n=5). An AT6.1 prostate tumor was implanted subcutaneously in the hind limb of the mouse. 1 mmol/kg of VO(acac)₂ was administered through a catheter placed in the tail vein. The VO(acac)₂ was dissolved in dimethyl sulfoxide and the resulting solution was diluted with saline. The final concentration of DMSO was about 5% by volume. The solution was about 10 mM VO(acac)₂.

The pH, pCO₂, HCO₃⁻, TCO₂, serum O₂, Na⁺, K⁺, Ca²⁺, glucose, blood urea nitrogen (BUN), Hematocrit (Hct) and hemoglobin (Hb) were monitored using an i-STAT analyzer (East Windsor, N.J.) by taking ˜30 μL blood samples over a period of three days. The observed values fell within the normal range specified in the literature.

### Example 6

### In Vivo Imaging of Rats Bearing Highly Invasive Metastatic AT6.1 Tumors

MRI studies were performed using a 4.7 Tesla Bruker scanner with multi-slice echo-planner spectroscopic imaging (EPSI). The scanning parameters were as follows: TR/TE of 800/80 msec; field of view 40 mm; array size of 256×256; slice thickness of 0.5 mm; and, an average of 1.

First, high-resolution reference images were acquired by conventional spin echo and gradient echo pulse sequences using multiple slices. These reference images provided anatomic reference information and were used to accurately measure tumor volume. Then multi-slice (usually 3 slices) echo-planner spectroscopic imaging (EPSI) with repetition time (TR) of 800 msec and echo time (TE) of 80 msec were acquired. Acquisition of MRI data using high-resolution EPSI pulse sequence may also be used. These sequence images were acquired with spatial resolution of 200 micron in-plane in ˜500 micron slices and spectral resolution of 2 Hz. These images were acquired at the same spatial resolution as conventional images but provided increased sensitivity to contrast media effects.

A Dunning AT6.1 prostate tumor was implanted subcutaneously in the hind limb of a Copenhagen rat. The tumor was monitored and imaging began when the tumor reached a size of 5-10 mm. The VO²⁺-based contrast agent was dissolved in dimethyl sulfoxide and the resulting solution was diluted with saline. The final concentration of DMSO was about 5% by volume. The solution was about 10 mM VO²⁺-based contrast agent.

After 6 to 8 pre-contrast MR images were obtained as a reference, 0.15 mmol/kg of VO(acac)₂ was injected through a catheter placed in the tail vein of the rat over 5 minutes. During MR imaging, the rats were anesthetized using isofluorane mixed with air and oxygen flowing through a mask. Exhaust gases were eliminated through an F-Air canister attached to the mask set up. Temperature was monitored continuously by a rectal thermometer and controlled with a warm water blanket. Heart rate, respiration rate, and blood pressure were monitored using an optical detector system from SA Instruments (Stony Brook, N.Y.). To minimize motion artifacts, rats were wrapped in co-flex and taped to a Plexiglass board. The leg was secured through the coil in a plastic tube and taped to the cradle.

Post-contrast EPSI scans were acquired for about 4 hours. Each EPSI scan took about 3.5 minutes. Images were used in which the intensity was proportional to the peak height of the water resonance in each voxel. The images are sensitive to both T₁ and T₂* changes following contrast agent injection. Pre-contrast images were compared with post-contrast images. These images were also compared to those obtained with Gd-DTPA in separate experiments.

FIG. 6 shows a high-resolution pre-contrast image (panel A), control images ([pre-contrast]−[pre-contrast]) (panel B), and difference ([post-contrast]−[pre-contrast]) images (up to T=2 hr after the administration of VO(acac)₂ (panel C). As shown in the panels of FIG. 6, some parts of the tumor became brighter and some parts became darker as a function of time, indicating accumulation of the contrast agent in the tumor. Tumor regions where intensity decreased suggests that the vanadyl-based contrast agent is being concentrated in micro environments (inside blood vessels or inside cells) causing gradients in magnetic susceptibility and thus decreases in T₂*. In other regions, the vanadyl-based contrast agent caused positive enhancement suggesting more homogeneous distribution leading to T₁ decreases.

### Example 7

### Contrast Media Dynamics in Rats Bearing AT6.1 Tumors

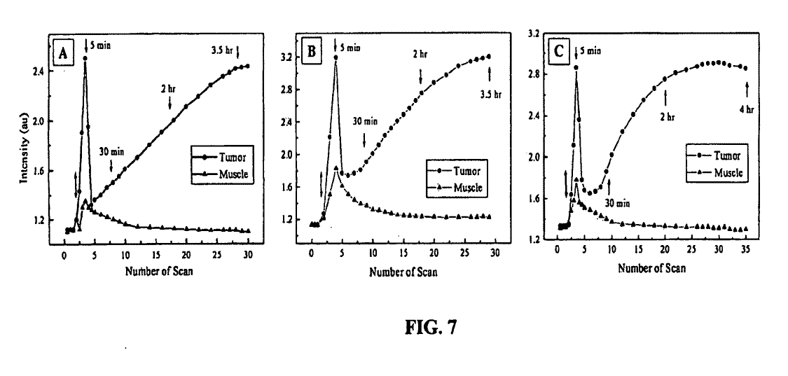

FIG. 7 shows the contrast media dynamics in representative regions of interest from five in vivo MRI experiments with rats bearing AT6.1 tumors.

A Dunning AT6.1 prostate tumor was implanted subcutaneously in the hind limb of a Copenhagen rat. The tumor was monitored and imaging began when the tumor reached a size of 5-10 mm. The VO²⁺-based contrast agent was dissolved in dimethyl sulfoxide and the resulting solution was diluted with saline. The final concentration of DMSO was about 5% by volume. The solution was about 10 mM VO²⁺-based contrast agent.

0.15 mmol/kg of VO(acac)₂ was injected through a catheter placed in the tail vein of the rat over 5 minutes. During MR imaging, the rats were anesthetized using isofluorane mixed with air and oxygen flowing through a mask. Exhaust gases were eliminated through an F-Air canister attached to the mask set up. Temperature was monitored continuously by a rectal thermometer and controlled with a warm water blanket. Heart rate, respiration rate, and blood pressure were monitored using an optical detector system from SA Instruments (Stony Brook, N.Y.). To minimize motion artifacts, rats were wrapped in co-flex and taped to a Plexiglass board. The leg was secured through the coil in a plastic tube and taped to the cradle.

The plots of relative signal intensity versus time support the hypothesis that VO(acac)₂ was selectively taken up by cancer cells based on contrast media dynamics. In all five experiments, very different dynamics were observed in tumor as compared to muscle. In muscle there was enhancement during the 5-minute injection period, followed by a fairly rapid decrease in enhancement after the injection, consistent with clearance of the VO(acac)₂ from blood by liver and kidneys. In many tumor regions, particularly regions near the tumor rim, there was initially strong enhancement just as in muscle, with higher maximum amplitude than in muscle, probably due to dense vasculature. At the end of the contrast agent injection, there was a transient decrease consistent with clearance from blood, and paralleling the decrease in muscle. However, after an initial decrease during the clearance phase, intensity increased continuously and significantly in many tumor areas, as shown in FIG. 7. Even though blood level was relatively low, as indicated by the signal in muscle, the tumor uptake was persistent. This is consistent with sequestration of VO²⁺ inside of cancer cells.

Due to the persistent uptake by the tumor, VO(acac)₂ enhanced specific regions of tumors selectively with relatively little enhancement in muscle for a long time after injection, as seen in FIG. 7. Enhancements were typically greater than 50% and often as high as 100%. Typical changes in signal intensity following injection of gadolinium-based low molecular weight contrast agents are very different, since Omniscan (Gd-agent) clears from the tumor much more rapidly than VO(acac)₂. This was what would be expected if VO(acac)₂ was taken up selectively by cancer cells in some tumor regions but not in muscle. The uptake and clearance of VO(acac)₂ from muscle and tumor is compared in the plots of signal intensity vs. time shown in FIG. 7. This suggests preferential leakage from tumor blood vessels and is also consistent with uptake of vanadyl into tumor cells. The results showed that VO(acac)₂ provides excellent T₁ and T₂* contrast.

### Example 8

### Concentration of VO(acac)₂ in Tumor and Muscle Tissue Using Atomic Absorption Spectroscopy

AT6.1 prostate tumors were implanted subcutaneously in the hind limbs of nude mice. The tumors were monitored and allowed to grow to about 6-8 mm over a period of about 2 weeks. The VO²⁺-based contrast agent was dissolved in dimethyl sulfoxide and the resulting solution was diluted with saline. The final concentration of DMSO was about 5% by volume. The solution was about 10 mM VO²⁺-based contrast agent.

VO(acac)₂ was added slowly to the tail vein of each mouse using a syringe infusion pump (Harvard Apparatus, South Natick, Mass.). A total of 100 μL of solution were added in 5 minutes (flow rate=20 μL/minute) for a total dosage of about 0.15 mmol/kg. All mice were kept in a cage and monitored after injection. The mice were then sacrificed at various times.

Tissue samples of the tumors and muscles were removed after a mouse was sacrificed. The tissue samples were weighed and then digested by wet digestion using H₂O₂ and HNO₃. Atomic absorption measurements were carried out using Perkin Elmer AAnalyst 300 with an HGA 800 furnace. Control sets (n=3) were free of contrast agent. Measurements were based on standard curves of known vanadyl concentration with a correlation coefficient of 0.99820.

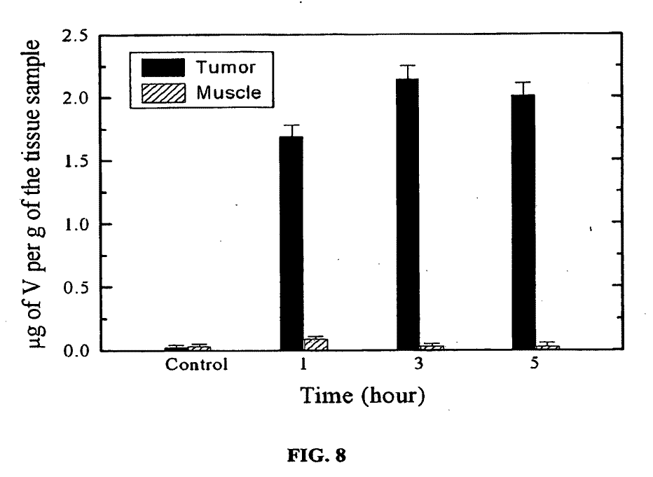

As shown in FIG. 8, the vanadyl concentration in tumor tissue was greatest at about 3 hours. However, even at 5 hours the vanadyl concentration remained relatively high. This observation is consistent with previous data, such as that shown in FIG. 8, wherein accumulation of the vanadyl contrast agent in the tumor and surrounding muscles increased with time. Therefore, these results highlight that vanadyl-based contrast agents accumulated in highly glycolytic cells and will specifically enhance these cells in MR imaging.

### Example 9

### Administering Contrast Agent to Patient and Imaging Process

MRI studies are performed on a patient intravenously injected with 0.15 mmol/kg of VO(acac)₂. The MRI studies are performed using a 4.7 Tesla Bruker scanner with multi-slice EPSI. High-resolution reference images are acquired by conventional spin echo and gradient echo pulse sequences using multiple slices. These reference images provide anatomical reference information and are used to accurately measure tumor volume. Then multi-slice (usually 3 slices) EPSI with a TR of 800 msec and TE of 80 msec are acquired. Acquisition of MRI data using high-resolution EPSI pulse sequence can also be used. The sequence images are acquired with a spatial resolution of 200 micron in-plane in ˜500 micron slices and a spectral resolution of 2 Hz.

Images are used in which the intensity is proportional to the peak height of the water resonance in each voxel. The images are sensitive to both T₁ and T₂* changes following contrast agent injection. After several pre-contrast MRI images are obtained as a reference, 0.15 mmol/kg of the VO(acac)₂ contrast agent formulation (VO(acac)₂ added to an aqueous buffered solution comprising 0.01 M HEPES and 0.15 M NaCl at a phi of 7.4 and passed through a sterile, syringe-driven filter unit containing 0.22 micron filter unit) is intravenously injected into a patient over 5 minutes. The Post-contrast EPSI scans are completed at 0.5, 1, 1.5 and 2 hours after injection. Pre-contrast images are compared with post-contrast images.

1. A method of in vivo imaging a population of cells in a subject comprising:
administering a VO²⁺-based contrast agent to the subject; and
monitoring distribution of the VO²⁺-based contrast agent in the subject using magnetic resonance imaging.

2. The method of claim 1, wherein a first magnetic resonance image is obtained prior to administering the VO²⁺-based contrast agent and a second magnetic resonance image is obtained after the VO²⁺-based contrast agent is administered and the first and the second magnetic resonance images are compared.

3. The method of claim 1, wherein the population of cells comprises an organ.

4. The method of claim 1, wherein the population of cells is selected from the group consisting of pancreas, brain, lung, kidney, thyroid, genitourinary, colorectal, gastrointestinal, liver, central nervous system, peripheral nervous system, prostate, heart and breast.

5. The method of claim 1, wherein the population of cells comprises a tumor.

6. The method of claim 1, wherein the population of cells comprises cancer cells.

7. The method of claim 1, wherein the subject is a human.

8. The method of claim 1, wherein the VO²⁺-based contrast agent comprises at least one organic ligand chelated to a VO²⁺ ion.

9. The method of claim 8, wherein at least one organic ligand is bound to the VO²⁺ ion through at least one heteroatom selected from the group consisting of oxygen and sulfur.

10. The method of claim 8, wherein at least one organic ligand is bidentate.

11. The method of claim 8, wherein at least one organic ligand contains at least one substitutent having an exchangeable proton.

12. The method of claim 11, wherein the substituent is selected from the group consisting of amino, thiol, and hydroxyl.

13. The method of claim 8, wherein at least one organic ligand is selected from the group consisting of acetylacetone, acetoacetamide, malonamide, 3-hydroxy-2-methyl-4-pyrone and 2-mercaptopyridine-N-oxide.

14. The method of claim 1, wherein the VO²⁺-based contrast agent has the formula:
VO[(Lₘ)ₙ₁(L_{b})ₙ₂(Lₜ)ₙ₃(Lₑ)ₙ₄]
wherein
Lₘ is a monodentate organic ligand;
L_{b} is a bidentate organic ligand;
Lₜ is a tridentate organic ligand; and
Lₑ is a tetradentate organic ligand; and
wherein
n₁ is 0, 1, 2, 3 or 4;
n₂ is 0, 1 or 2;
n₃ is 0 or 1; and
n₄ is 0 or 1;
such that
when n₁=4, then n₂, n₃ and n₄=0;
when n₁=2 and n₂=2, then n₃ and n₄=0;
when n₁=1 and n₃=1, then n₂ and n₄=0;
when n₂=2 then n₁, n₃ and n₄=0; and
when n₄=1 then n₁, n₂ and n₃=0.

15. The method of claim 14, wherein the VO²⁺-based contrast agent is selected from the group consisting of
VO(Lₘ¹Lₘ²Lₘ³Lₘ⁴),
VO(Lₘ¹Lₘ²L_{b}¹),
VO(L_{b}¹L_{b}²),
VO(LₜLₘ¹), or
VO(Lₑ)
wherein Lₘ¹, Lₘ², Lₘ³ and Lₘ⁴ are the same or different monodentate ligands selected from the group consisting of a heteroalkyl group, a substituted heteroalkyl group, a substituted alkyl group, a substituted aromatic group, a substituted heteroaromatic group, a substituted carbocyclic group, a substituted heterocyclic group, wherein each ligand is bound to vanadium through a coordinating atom selected from the group consisting of oxygen and sulfur; and
wherein L_{b}¹ and L_{b}² are the same or different bidentate ligands selected from the group consisting of a heteroalkyl group, a substituted heteroalkyl group, a substituted alkyl group, a substituted aromatic group, a substituted heteroaromatic group, a substituted carbocyclic group, a substituted heterocyclic group, wherein each ligand is bound to vanadium through two coordinating atoms selected from the group consisting of oxygen, sulfur and combinations thereof; and
wherein Lₜ is a tridentate ligand selected from the group consisting of a heteroalkyl group; a substituted heteroalkyl group, a substituted alkyl group, a substituted aromatic group, a substituted heteroaromatic group, a substituted carbocyclic group, a substituted heterocyclic group, wherein each ligand is bound to vanadium through three coordinating atoms selected from the group consisting of oxygen, sulfur and combinations thereof; and
wherein Lₑ is a tetradentate ligand selected from the group consisting of a heteroalkyl group, a substituted heteroalkyl group, a substituted alkyl group, a substituted aromatic group, a substituted heteroaromatic group, a substituted carbocyclic group, a substituted heterocyclic group, wherein each ligand is bound to vanadium through four coordinating atoms selected from the group consisting of oxygen, sulfur and combinations thereof.

16. The method of claim 15, wherein the VO²⁺-based contrast agent is
VO(L_{b}¹L_{b}²)
wherein L_{b}¹ and L_{b}² are the same or different bidentate ligands selected from the group consisting of a heteroalkyl group, a substituted heteroalkyl group, a substituted alkyl group, a substituted aromatic group, a substituted heteroaromatic group, a substituted carbocyclic group, a substituted heterocyclic group, wherein each ligand is bound to vanadium through two coordinating atoms selected from the group consisting of oxygen, sulfur and combinations thereof.

17. The method of claim 16, wherein at least one of L_{b}¹ and L_{b}² contains a substituent.

18. The method of claim 17, wherein the substituent is NH₂, SH or OH.

19. The method of claim 15, wherein the VO²⁺-based contrast agent is selected from the group consisting of bis(acetylacetonato)oxovanadium(1V), bis(acetylamido)oxovanadium(IV), bis(maltolato)oxovanadium(IV), bis(N-oxide-pyridine-2-thiolato)oxovanadium(IV), bis(acetylacetanato-bisNH₂)oxovanadium(IV) and combinations thereof.

20. The method of claim 1, wherein the VO²⁺-based contrast agent has a molecular weight of less than about 600.

21. The method of claim 1, wherein the VO²⁺-based contrast agent has a molecular weight of less than about 500.

22. The method of claim 1, wherein the VO²⁺-based contrast agent has a molecular weight of less than about 400.

23. The method of claim 1, wherein the VO²⁺-based contrast agent has a molecular weight of less than about 300.

24. The method of claim 1, wherein about 0.01 to about 0.20 mmol/kg body weight of VO²⁺-based contrast agent is administered to the subject.

25. The method of claim 1, wherein about 0.1 to about 0.15 mmol/kg body weight of the VO²⁺-based contrast agent is administered to the subject.

26. The method of claim 1, wherein the VO²⁺-based contrast agent is administered parenterally.

27. The method of claim 1, wherein the VO²⁺-based contrast agent is administered orally.

28. The method of claim 1, wherein the VO²⁺-based contrast agent is administered via inhalation.

29. The method of claim 1, wherein the VO²⁺-based contrast agent is administered directly into the population of cells being imaged.

30. The method of claim 1, wherein the VO²⁺-based contrast agent is administered over a time period of about 0.5 to about 2 hours.

31. The method of claim 1, wherein the VO²⁺-based contrast agent is administered with a physiologically acceptable carrier.

32. A method of detecting cancer cells in a subject comprising:
obtaining a first magnetic resonance image of a region of the subject;
administering a VO²⁺-based contrast agent to the subject;
obtaining a second magnetic resonance image of the region; and
comparing the first and second magnetic resonance images.

33. A method of detecting cancer cells in a subject comprising:
administering a VO²⁺-based contrast agent to the subject;
obtaining a magnetic resonance image of the subject; and
identifying regions of enhanced contrast in the magnetic resonance image.

34. The method of claim 33, wherein the regions of enhanced contrast are determined as a function of time.

35. A method of enhancing magnetic resonance imageability of a subject, comprising administering to the subject an amount of a VO²⁺-based contrast agent effective to enhance a magnetic resonance image.

36. The method of claim 35 further comprising obtaining a magnetic resonance image of the subject, wherein the image has enhanced contrast.

37. A composition comprising:
a VO²⁺-based contrast agent; and
a physiologically acceptable organic solvent; and
a physiologically acceptable buffer.

38. A composition comprising:
a VO²⁺-based contrast agent; and
a physiologically acceptable carrier,
wherein the VO²⁺-based contrast agent is present in a concentration of at least about 1 mM.

39. A compound having the formula: [chemical structure]
